(19) United States
(12) Patent Application Publication
(10) Pub. No.: US 2024/0074914 A1
(43) Pub. Date: Mar. 7, 2024

(54) PASSIVE NEGATIVE PRESSURE DRESSING

(71) Applicant: KCI Manufacturing Unlimited Company, Westmeath (IE)

(72) Inventors: Timothy Mark ROBINSON, San Antonio, TX (US); Thomas Alan EDWARDS, San Antonio, TX (US); John R. HARPER, San Antonio, TX (US); Christopher Brian LOCKE, San Antonio, TX (US)

(21) Appl. No.: 18/274,584

(22) PCT Filed: Jan. 20, 2022
(86) PCT No.: PCT/IB2022/050466

§ 371 Date: Jul. 27, 2023

Related U.S. Application Data
(60) Provisional application No. 63/150,356, filed on Feb. 17, 2021.

(51) Int. Cl. A61F 13/12; A61F 13/02; A61M 1/00

(52) U.S. Cl. CPC A61F 13/122; A61F 13/0206; A61F 13/0216; A61F 13/0253; A61M 1/94

(57) ABSTRACT

An oxygen scavenging dressing includes a patient interface layer, an absorbent layer, a foam layer, an oxygen scavenging layer, and a film layer. The film layer is configured to seal with a patient's skin and define an inner volume within which the patient interface layer, the absorbent layer, the foam layer, and the oxygen scavenging layer are positioned.

### CROSS-REFERENCE TO RELATED APPLICATIONS

This application claims the benefit of priority to U.S. Provisional Application No. 63/150,356, filed on Feb. 17, 2021, which is incorporated herein by reference in its entirety.

### BACKGROUND

The present disclosure relates generally to dressings. More particularly, the present disclosure relates to dressings for areas of the body (e.g., the face) which require smaller, lower profile dressings. When a patient undergoes any sort of facial surgery or treatment that requires an incision be made, a dressing may be applied to protect the incision from infection and to facilitate healing of the incision. It may be advantageous to provide negative pressure to facilitate the healing of the incision. However, dressings that are configured for active negative pressure (e.g., using a pump and tubes) are often too large for facial areas.

### SUMMARY

One implementation of the present disclosure is an oxygen scavenging dressing, according to some embodiments. The oxygen scavenging dressing includes a patient interface layer, an absorbent layer, a foam layer, an oxygen scavenging layer, and a film layer. The film layer is configured to seal with a patient's skin and define an inner volume within which the patient interface layer, the absorbent layer, the foam layer, and the oxygen scavenging layer are positioned.

In some embodiments, the oxygen scavenging layer is configured to remove oxygen from within the inner volume of the oxygen scavenging dressing to draw a negative pressure within the oxygen scavenging dressing.

In some embodiments, the oxygen scavenging dressing is positioned on a patient's face.

In some embodiments, the film layer includes an adhesive along an underside configured to seal with the patient's skin.

In some embodiments, the patient interface layer abuts the patient's skin.

In some embodiments, the absorbent layer is positioned directly between the foam layer and the patient interface layer.

In some embodiments, the foam layer is positioned directly between the oxygen scavenging layer and the absorbent layer.

In some embodiments, the oxygen scavenging layer includes a chemical substance configured to remove oxygen from within the inner volume.

In some embodiments, the chemical substance is an iron compound.

In some embodiments, the oxygen scavenging layer includes two or more sections configured to be selectively deployed to customize a negative pressure capability of the oxygen scavenging dressing.

Another implementation of the present disclosure is an oxygen scavenging dressing, according to some embodiments. In some embodiments, the oxygen scavenging dressing includes an oxygen scavenging layer, one or more additional layers, and a film layer. The film layer is configured to seal with a patient's skin and define an inner volume within which the oxygen scavenging layer and the one or more additional layers are positioned. The oxygen scavenging layer is configured to remove oxygen from within the inner volume of the oxygen scavenging dressing to draw a negative pressure within the oxygen scavenging dressing.

In some embodiments, the one or more additional layers include a patient interface layer, an absorbent layer, and a foam layer.

In some embodiments, the oxygen scavenging dressing is positioned on a patient's face.

In some embodiments, the film layer includes an adhesive along an underside configured to seal with the patient's skin.

In some embodiments, the patient interface layer abuts the patient's skin.

In some embodiments, the absorbent layer is positioned directly between the foam layer and the patient interface layer.

In some embodiments, the foam layer is positioned directly between the oxygen scavenging layer and the absorbent layer.

In some embodiments, the oxygen scavenging layer includes a chemical substance configured to remove oxygen from within the inner volume.

In some embodiments, the chemical substance is an iron compound.

In some embodiments, the oxygen scavenging layer includes two or more sections configured to be selectively deployed to customize a negative pressure capability of the oxygen scavenging dressing.

Another implementation of the present disclosure is a method for passively drawing a negative pressure at a dressing, according to some embodiments. The method includes providing a dressing including an oxygen scavenging material, a film layer, and one or more other layers. The method also includes adhering the dressing to a patient's skin over an incision or a wound to define a sealed inner volume. The method also includes drawing a negative pressure within the sealed inner volume by scavenging oxygen within the sealed inner volume at the oxygen scavenging material.

In some embodiments, the method includes selectably fluidly coupling one or more additional sections of oxygen scavenging material with the sealed inner volume to further decrease the negative pressure.

In some embodiments, the method includes selectively deploying two or more sections to customize a negative pressure capability of the dressing.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a sectional view of a passive negative pressure wound dressing in a first state, according to an exemplary embodiment;

FIG. 2 is a sectional view of the passive negative pressure wound dressing of FIG. 1 in a second state, according to an exemplary embodiment;

FIG. 3 is a graph of vacuum pressure with respect to time for different passive dressings, according to an exemplary embodiment; and

FIG. 4 is a flow diagram of a process for providing and using a passive negative pressure dressing, according to an exemplary embodiment.

FIG. 5 is a sectional view of the passive negative pressure wound dressing of FIG. 1, including two pockets of oxygen scavenging material, according to an exemplary embodiment.

FIG. 6 is a top view of the passive negative pressure wound dressing of FIG. 5, according to an exemplary embodiment.

### DETAILED DESCRIPTION

### Overview

Referring generally to the FIGURES, a passive negative pressure dressing is shown, according to an exemplary embodiment. In this context, “passive” is intended to include dressing capable of operating at a negative pressure without conventional vacuum pumps and tubing connected to the dressing. The passive negative pressure dressing includes a film layer, an oxygen scavenging layer, and one or more other layers. The film layer may be a dressing or an outer layer that is configured to cover the oxygen scavenging layer and the one or more other layers. The film layer may define a sealed inner volume within which the oxygen scavenging layer and the one or more other layers are positioned. The one or more other layers may include a patient interface layer that is configured to interface with, engage, abut, or directly contact the patient's skin, an absorbent layer that abuts the patient interface layer, and a foam layer that abuts the absorbent layer. The oxygen scavenging layer can include an iron compound that is configured to consume or decrease an amount of oxygen within the inner volume to draw a negative pressure.

### Passive Negative Pressure Dressing

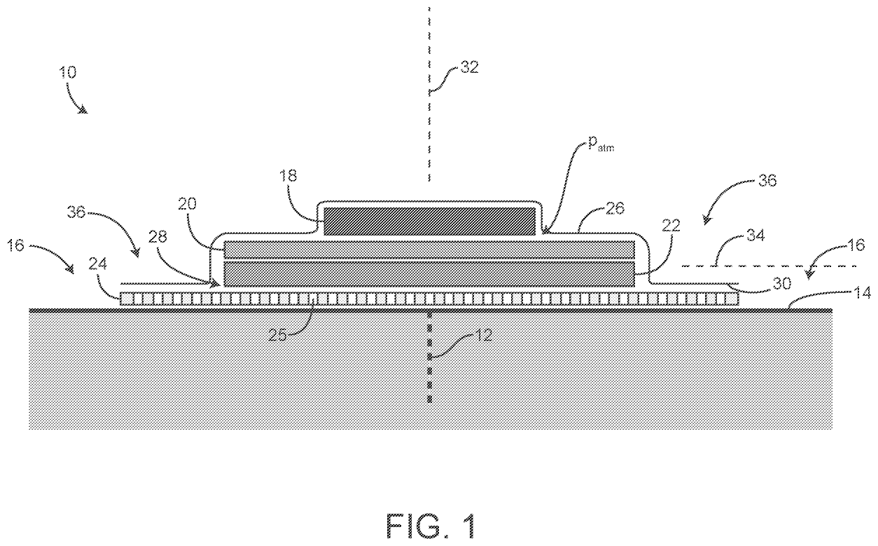

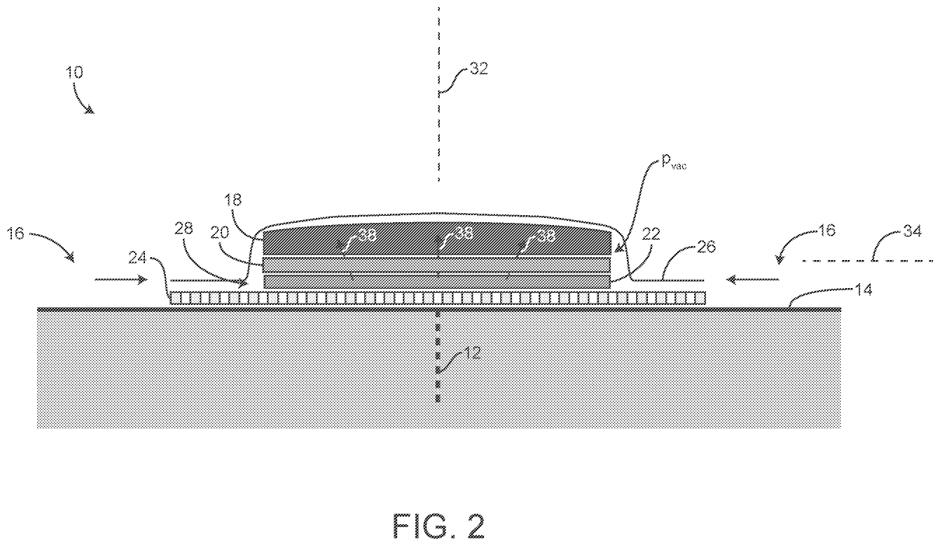

Referring to FIGS. 1-2, a passive negative pressure dressing, an oxygen scavenging negative pressure dressing, a pump-less negative pressure dressing, etc., shown as oxygen scavenging dressing 10 is shown, according to an exemplary embodiment. Oxygen scavenging dressing 10 can include a polyurethane film, shown as film 26, an oxygen scavenging layer 18, a foam layer 20, an absorbent layer 22, and a patient interface layer 24. The oxygen scavenging layer 18, the foam layer 20, the absorbent layer 22, and the patient interface layer 24 may be arranged relative to each other as shown in FIGS. 1-2. Specifically, the oxygen scavenging layer 18, may be an outermost or upper layer, with the foam layer 20 positioned directly beneath the oxygen scavenging layer 18, between the oxygen scavenging layer and the absorbent layer 22. The foam layer 20 may be positioned between the oxygen scavenging layer 18 and the absorbent layer 22. The absorbent layer 22 may be positioned between the foam layer 20 and the patient interface layer 24.

Oxygen scavenging dressing 10 can have a size that is between 20 mm×50 mm to approximately 100 mm×100 mm. Oxygen scavenging dressing 10 can draw down to a vacuum or negative pressure of 50 mmHg to 150 mmHg. Oxygen scavenging dressing 10 may have a relatively high vertical or compressive stiffness with low creep, but high flexibility (e.g., along axis 34) to facilitate proper conformation to the patient's body contour. Oxygen scavenging dressing 10 can facilitate efficient transfer of vacuum to patient's tissue to encourage decompression and lateral closure of a wound or incision (e.g., incision 12). Oxygen scavenging dressing 10 can include slits or other forms that facilitate or permit shrinkage of the dressing 10 due to the generated vacuum or other forces.

The patient interface layer 24 can be configured to directly engage, abut, contact, seal with, etc., a patient's tissue (e.g., the patient's skin 14). The film 26 may completely cover all of the oxygen scavenging layer 18, the foam layer 20, the absorbent layer 22, and the patient interface layer 24. In some embodiments, the film 26 extends outwards beyond an outer periphery of the patient interface layer 24 and seals circumferentially or peripherally with the patient's skin 14 (e.g., around the outer periphery of the patient interface layer 24). The film 26 may also or alternatively seal directly with an outwards or exterior surface of the patient interface layer 24.

The oxygen scavenging dressing 10 substantially covers an incision 12 that extends into the patient's tissue. The incision 12 may be on a patient's face such that the oxygen scavenging dressing 10 is placed over the incision 12 as a face dressing. Advantageously, oxygen scavenging dressing 10 can be low profile, small, and lightweight so that oxygen scavenging dressing 10 can be used in locations such as the patient's face where discreteness is desired or required.

The oxygen scavenging dressing 10 can have a size (e.g., a footprint on the patient's skin 14) of between 20 mm×50 mm to approximately 100 mm×100 mm. The oxygen scavenging dressing 10 can be primarily used for surgical wounds located on the face (e.g., incision 12) but may also be used to treat other surgical and low exudating wounds.

The film layer 26 can be coated on an underside 30 (e.g., an incision facing side) with an adhesive that is configured to facilitate sealing between the film layer 26 and the patient's skin 14 (e.g., an exterior surface of the patient's skin 14) and/or an exterior surface (e.g., an outwards facing surface) of the patient interface layer 24. In some embodiments, the underside 30 is also configured to sealingly couple (e.g., through the adhesive) with exterior or outwards facing surfaces of the absorbent layer 22, the foam layer 20, and/or the oxygen scavenging layer 18.

The adhesive on the underside 30 of the film layer 26 may cover or coat a complete surface area of the underside 30. In some embodiments, the film layer 26 includes adhesive along only portions of the underside 30 of the film layer 26 (e.g., where the film layer 26 is expected to adhere to the patient's skin 14, the foam layer 20, the absorbent layer 22, or the oxygen scavenging layer 18). The adhesive may be an acrylic adhesive. The adhesive may serve two functions: first, the adhesive may seal contents (e.g., the oxygen scavenging layer 18, the foam layer 20, the absorbent layer 22, and the patient interface layer 24) inside of oxygen scavenging dressing 10, and second, the adhesive may aid in adhesion of the film 26 and oxygen scavenging dressing 10 with the patient's skin 14 (e.g., periwound tissue 16). For example, the film layer 26 may be an Inspire 2301 film layer. The film layer 26 can define an inner volume 28 between the film layer 26 and the patient's skin 14.

The oxygen scavenging layer 18 can be positioned directly below or adjacent to the film layer 26. The oxygen scavenging layer 18 may include a chemical substance that is configured to reduce or completely remove oxygen from within the inner volume 28. As the oxygen scavenging layer 18 removes oxygen from within the inner volume 28, inner volume 28 may decrease, thereby resulting in a reduced, negative, or vacuum pressure within inner volume 28. An amount, size, or rating of the oxygen scavenging layer 18 may determine a vacuum range that results at a wound site that oxygen scavenging dressing 10 is configured to treat (e.g., incision 12). In some embodiments, an amount of the chemical substance or a concentration of the chemical substance of the oxygen scavenging layer 18 determines how much of the oxygen is removed from inner volume 28, or a magnitude of the vacuum pressure that results within inner volume 28.

The chemical substance in the oxygen scavenging layer 18 can be iron or an iron compound such as ferrous iron, ascorbic acid, sulphites, or pyrogallol. In some embodiments, the chemical substance includes at least one of ferrous iron, ascorbic acid, sulphites, or pyrogallol. For example, the chemical substance may be iron or iron oxide (FeO) that is mixed with sodium chloride and optionally activated carbon. When the iron or iron oxide reacts or ‘rusts’ in the presence of moisture to form ferrous oxide, the iron or iron oxide absorbs oxygen from within the inner volume 28, thereby drawing a vacuum pressure within inner volume 28.

In some embodiments, the oxygen scavenging layer 18 includes an activated carbon and clay, and/or volatile materials such as water and alcohols. The volatile materials may be mixed with an absorbing material such as a super-absorber that is configured to swell. When the oxygen scavenging dressing 10 operates over time to draw a vacuum pressure within inner volume 28, the absorbing material may shrink as it loses the volatile materials, thereby causing a pressure drop.

The foam layer 20 can be manufactured from or include a closed cell foam material. The foam layer 20 can facilitate providing rigidity for the oxygen scavenging dressing 10 when the negative pressure is drawn within inner volume 28. In some embodiments, the foam layer 20 includes a pattern cut into it to facilitate or ensure lateral retraction (e.g., to facilitate retraction in a lateral direction along lateral axis 34) and provide rigidity in a longitudinal direction (e.g., along longitudinal axis 32). The patterns can be cut to extend in a direction along longitudinal axis 32. In this way, when the negative pressure is drawn within inner volume 28, the foam layer 20 can retract along lateral axis 34 but provide rigidity along the longitudinal axis 32 (e.g., such that retraction along the longitudinal axis 32 is minimal). Foam layer 20 can be configured to retract or reduce in length a greater amount along the lateral axis 34 than along the longitudinal axis 32. In this way, foam layer 20 provides rigidity for oxygen scavenging dressing 10 in a vertical direction (e.g., along the longitudinal axis 32, in a direction perpendicular to the patient's skin 14) and is bias to retract or decrease in length in a horizontal direction (e.g., along the lateral axis 34, in a direction along the patient's skin 14). When a negative or vacuum pressure is drawn within inner volume 28 (e.g., by reaction of the chemical substance in oxygen scavenging layer 18), foam layer 20 facilitates retraction or compression of oxygen scavenging dressing 10 in the longitudinal or horizontal direction (e.g., along lateral axis 34), thereby pulling together edges of incision 12. Foam layer 20 may be a closed cell foam such as ZoteFoam (e.g., Evazote® EV50, ethylene vinyl acetate).

Referring still to FIG. 1, oxygen scavenging dressing 10 includes absorbent layer 22, according to some embodiments. Absorbent layer 22 may be positioned between foam layer 20 and patient interface layer 24. Absorbent layer 22 can be positioned and configured so that if any wound fluid, exudate, liquid, etc., is/are exuded, emitted, output, discharged, etc., from the incision 12 as negative or vacuum pressure is drawn within oxygen scavenging dressing 10, the wound fluid is absorbed and retained in absorbent layer 22 (e.g., a fluid absorbent layer). In some embodiments, absorbent layer 22 is positioned directly beneath or below the foam layer 20. Absorbent layer 22 may be positioned directly adjacent or contacting the patient's skin 14, or may be positioned directly adjacent or contacting patient interface layer 24.

The absorbent layer 22 may be a super absorbent polymer (SAP) T-bag (e.g., including a wicking outer layer) with SAP enclosed within the outer layer. The wicking outer layer may be Libeltex TDL2 80 gsm, wicking microfiber (Evolon, Freudenberg), or an absorbent foam layer such as hydrophilic polyurethane foam (Medisponge, Porex; Freudenberg).

Referring still to FIG. 1, oxygen scavenging dressing 10 includes patient interface layer 24, according to some embodiments. Patient interface layer 24 may be a layer that engages, contacts, abuts, or otherwise interfaces directly with the patient's skin 14 (e.g., proximate the incision 12. Patient interface layer 24 can facilitate a reliable seal around the edge of the incision 12 (e.g., between the patient interface layer 24 and the patient's skin 14). Patient interface layer 24 can include one or more perforations 25 (e.g., openings, holes, slots, etc.) to allow fluid exuded from the incision 12 to pass through the patient interface layer 24 and reach the absorbent layer 22. Perforations 25 can facilitate allowing controlled or certain sections of the underside 30 of film 26 to engage and seal with the patient's skin 14 around the incision 12 (e.g., at outer areas 36). In some embodiments, an underside or skin-facing side of patient interface layer 24 includes an adhesive that is configured to adhere with the patient's skin 14. Patient interface layer 24 can facilitate a seal at incision 12 or the wound site for oxygen scavenging dressing 10 to function properly. Additionally, since film 26 only sealingly couples or adheres with the patient's skin 14 through perforations 25, this may reduce trauma or removal force when oxygen scavenging dressing 10 is removed and/or replaced.

In some embodiments, oxygen scavenging dressing 10 includes an additional wound interface layer. The additional wound interface layer may be positioned between the patient interface layer 24 and the patient's skin 14. The additional wound interface layer may be designed to provide additional support for healing of surgical wounds. The additional wound interface layer can be configured to wick moisture from the wound (e.g., from incision 12) to prevent maceration of incision 12 and periwound areas 16. In some embodiments, the additional wound interface layer is manufactured from or includes a Milikan Non-Woven material.

Referring to FIGS. 1-2, oxygen scavenging dressing 10 is shown transitioned between an initial state (shown in FIG. 1) and when oxygen scavenging dressing 10 has operated to absorb oxygen from within the inner volume 28 of oxygen scavenging dressing 10. As shown in FIGS. 1-2, oxygen scavenging layer 18 reacts with oxygen within inner volume 28 to absorb the oxygen. This results in oxygen scavenging layer 18 creating a vacuum pressure originating at oxygen scavenging layer 18. The vacuum pressure may draw fluid, oxygen, gas, etc., from lower in the oxygen scavenging dressing 10 to the oxygen scavenging layer 18 (e.g., upwards, as indicated by arrows 38). The fluid can be drawn from the patient's skin 14 or the incision 12 to the oxygen scavenging layer 18 through the absorbent layer 22, the foam layer 20, and to the oxygen scavenging layer. The oxygen scavenging layer 18 may increase in size or volume as it absorbs liquid, exuded wound fluid, oxygen, etc., from within the inner volume 28 of oxygen scavenging dressing 10 as shown in FIGS. 1-2.

As the oxygen scavenging dressing 10 (or more specifically oxygen scavenging layer 18) absorbs liquid, fluid, oxygen, etc., that is within inner volume 28 of oxygen scavenging dressing 10, a compressive force is produced along lateral axis 34. The compressive force can facilitate providing a compressive or a closing force to incision 12. Advantageously, opposite surfaces or edges of incision 12 may be pressed into compression with each other as the oxygen scavenging dressing 10 operates to absorb oxygen and produce the vacuum pressure within inner volume 28. This can facilitate improved healing of incision 12 (e.g., reduce an amount of time for incision 12 to heal sufficiently).

In addition to apposition forces being developed in oxygen scavenging dressing 10 due to gas absorption or liquid loss (e.g., at oxygen scavenging layer 18), resulting shrinkage may be used to generate mechanical displacement when coupled to the periwound 16 (e.g., the patient's skin 14). One advantage of oxygen scavenging dressing 10 is that it reduces a need for a high pneumatic seal as discreet anchor points on the periwound enable mechanical shrinkage forces to be transferred to these locations.

### Pressure Drawdown

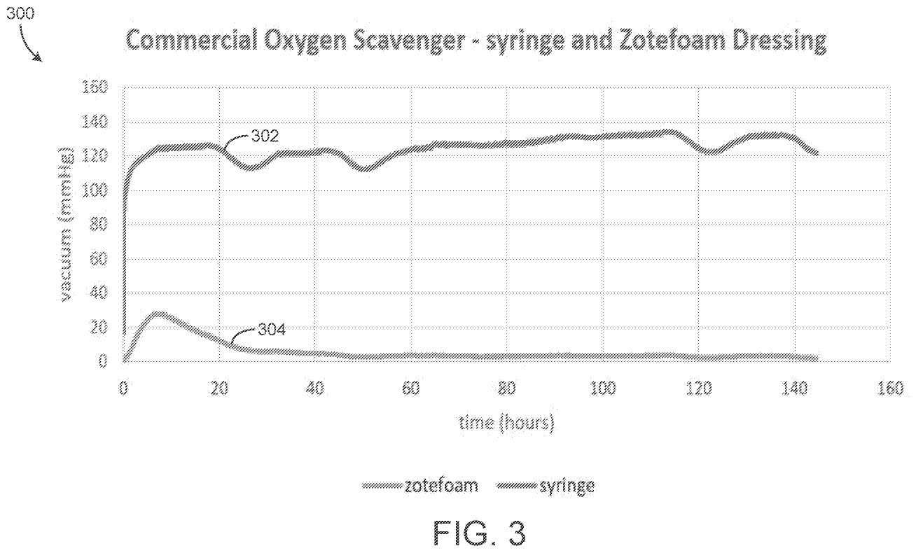

Referring particularly to FIG. 3, a graph 300 is shown that illustrates pressure drawdown (e.g., vacuum pressure in mmHg, the Y axis) over time (e.g., hours, the X-axis). Graph 300 includes series 302 and series 304. Series 302 illustrates pressure drawdown for a syringe dressing while series 304 illustrates pressure drawdown for a Zotefoam dressing. As shown in graph 300, the syringe dressing (illustrated by series 302) may achieve a faster drawdown to a higher vacuum pressure (e.g., approximately 120 mmHg). The syringe dressing is also able to maintain a more constant negative pressure over time, whereas the Zotefoam dressing experiences decay over time. Depending on the application of the oxygen scavenging dressing 10, the syringe dressing or the Zotefoam dressing may be chosen and used. For example, for an application where a higher and more consistent negative or vacuum pressure is desired, the syringe dressing may be used. If a pressure decay is desired over time, the Zotefoam dressing may be used.

In some embodiments, different oxygen scavenging dressings 10 can be configured to draw down to different pressures (e.g., different vacuum pressures or different negative pressures) based on an amount, mass, weight, or volume of oxygen scavenging material that is present within the sealed inner volume 28. For example, based on a size, weight, density, mass, etc., of oxygen scavenging layer 18, and/or a size or volume of inner volume 28, oxygen scavenging dressing 10 may draw down to different negative pressures. In this way, a relationship between a relative amount of oxygen scavenging material and a volume of inner volume 28 may determine a negative pressure that is produced within inner volume 28 when oxygen scavenging layer 18 absorbs oxygen, gas, or fluid and draws down oxygen scavenging dressing 10.

Oxygen scavenging dressing 10 as described herein provides a dressing that passively generates a negative pressure at a wound site (e.g., inner volume 28). Oxygen scavenging dressing 10 uses natural mechanisms (e.g., oxidation) to provide negative pressure to the wound site (e.g., oxygen scavenging). Advantageously, oxygen scavenging dressing 10 reduces or completely removes a requirement to draw down negative pressure using pneumatic tubing and/or an electrical power source to generate negative pressure or apposition displacement. Advantageously, oxygen scavenging dressing 10 provides unobtrusive therapy delivery without a need for a sealed conduit or tubing. Further, oxygen scavenging dressing 10 does not require a complicated and costly therapy system (e.g., a negative pressure device) and is a passively driven system. Oxygen scavenging dressing 10 may be a simple to use dressing (requiring only application or adherence of the dressing) that may even be used or applied by a patient without caregiver assistance. This can reduce or remove the need for face to face consultations with care givers, which may provide cost savings.

### Dressing Activation

Referring to FIGS. 1-2, oxygen scavenging dressing 10 may include multiple pockets, sections, or portions of oxygen scavenging layer 18. The different sections of oxygen scavenging layer 18 can be selectably fluidly coupled with inner volume 28. For example, each section of the oxygen scavenging layer 18 may be positioned within a corresponding pocket or inner volume. Each section can be selectably fluidly coupled with the inner volume 28 by removing a barrier (e.g., an adhesive film, a polyurethane film, a removable film, etc.) to fluidly couple the inner volume of the pocket that contains the section of the oxygen scavenging material with inner volume 28 of the entire oxygen scavenging dressing 10.

In some embodiments, several sections of the oxygen scavenging layer 18 or the oxygen scavenging material are provided in oxygen scavenging dressing 10. For example, three sections or portions of the oxygen scavenging layer 18 may be provided which can each be fluidly coupled with the inner volume 28. The three sections or portions of the oxygen scavenging layer 18 can be selectably fluidly coupled with the inner volume 28 in series or in parallel. For example, each of the three sections may be independently selectably fluidly coupled with inner volume 28 (e.g., parallel). In another embodiment, a first of the three sections can be selectably fluidly coupled with the inner volume 28, while a second of the three sections can only be fluidly coupled with the inner volume 28 if the first section is fluidly coupled with the inner volume 28. Similarly, the third section may be fluidly coupled with the inner volume 28 only if the first section and the second section are fluidly coupled with the inner volume 28.

In some embodiments, fluidly coupling the different sections of the oxygen scavenging layer 18 or the oxygen scavenging material is achieved by a caregiver (e.g., before or after providing the oxygen scavenging dressing 10 to the user's skin 14). The caregiver may remove different adhesive layers to fluidly couple a number of the sections of the oxygen scavenging layer 18 to the inner volume 28. A number of sections of the oxygen scavenging layer 18 that are fluidly coupled with the inner volume 28 may determine a vacuum pressure or negative pressure that is produced within inner volume 28. For example, if three sections of oxygen scavenging material are provided, the oxygen scavenging dressing 10 can be transitionable between low, medium, and high modes, each mode of which corresponds to a different negative pressure or vacuum pressure within inner volume 28. Fluidly coupling only the first of the three sections of the oxygen scavenging material with inner volume 28 may result in oxygen scavenging dressing 10 drawing down to a first negative or vacuum pressure according to a low mode. Fluidly coupling both the first and the second of the three sections of the oxygen scavenging material with inner volume 28 may result in oxygen scavenging dressing 10 drawing down to a second negative or vacuum pressure according to a medium mode. Fluidly coupling the first, second, and third of the three sections of the oxygen scavenging material with inner volume 28 may result in oxygen scavenging dressing 10 drawing down to a third negative or vacuum pressure according to a high mode. It should be understood that while the example described herein uses only three sections of oxygen scavenging material so that the oxygen scavenging dressing 10 can operate or draw down according to a low, medium, or high mode, any number of oxygen scavenging sections can be used to provide any number of negative pressure or vacuum drawdown modes.

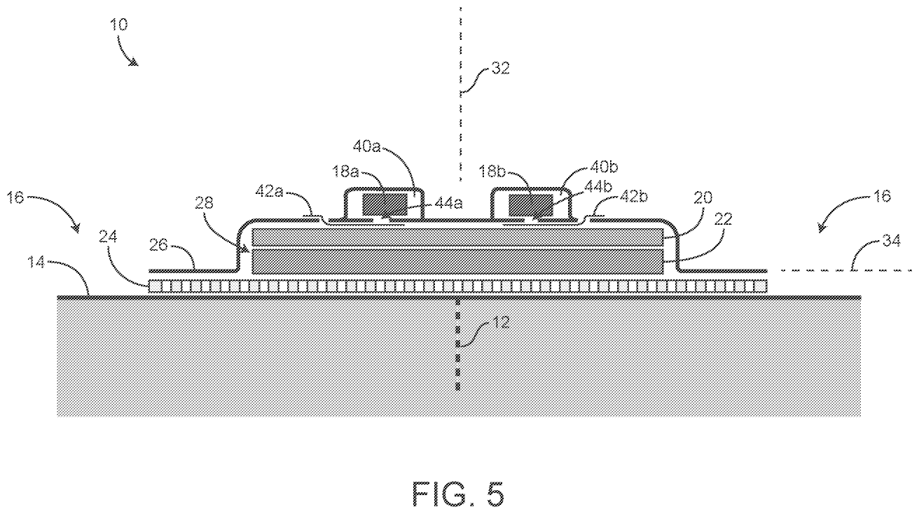

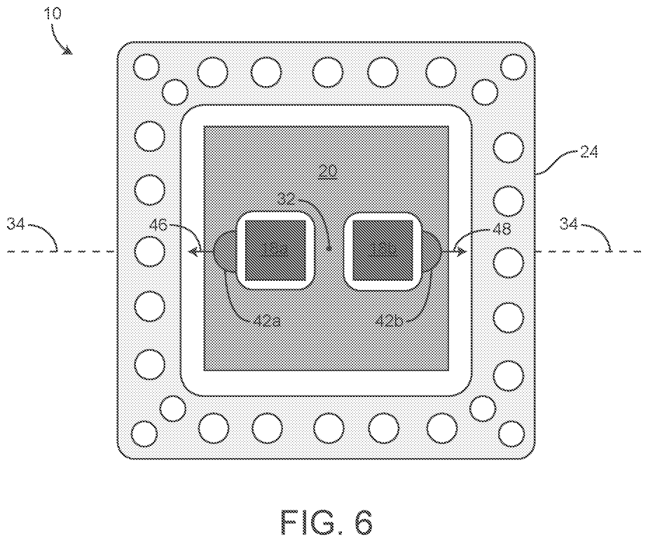

Referring particularly to FIGS. 5-6, oxygen scavenging dressing 10 with multiple sections, pockets, volumes, etc., of oxygen scavenging material or layers is shown, according to another embodiment. Oxygen scavenging dressing 10 as described herein with reference to FIG. 10 can be the same as or similar to oxygen scavenging dressing 10 as described with reference to FIGS. 1-2. Oxygen scavenging dressing 10 includes a first inner volume, a first section, a first portion, etc., shown as first pocket 40a, and a second inner volume, a second section, a second portion, etc., shown as second pocket 40b. First pocket 40a includes an oxygen scavenging layer, an oxygen scavenging portion, etc., shown as first oxygen scavenging member 18a and second pocket 40b includes an oxygen scavenging layer, an oxygen scavenging portion, etc., shown as second oxygen scavenging member 18b.

The first oxygen scavenging member 18a and the second oxygen scavenging member 18b can be selectively coupled with the inner volume 28 by removal of an adhesive member 42a and/or an adhesive member 42b. Specifically, the first pocket 40a can be fluidly coupled with the inner volume 28 through an opening 44a when the member 42a is removed. Similarly, the second pocket 40b can be fluidly coupled with the inner volume 28 through an opening 44b when the member 42b is removed. In this way, one or both of the first oxygen scavenging member 18a and the second oxygen scavenging member 18b can be fluidly coupled with the inner volume 28 to activate different amounts of draw down. For example, when the first oxygen scavenging member 18a is fluidly coupled with inner volume 28, a first vacuum or drawdown pressure may be achieved within inner volume 28. When both the first oxygen scavenging member 18a and the second oxygen scavenging member 18b are fluidly coupled with inner volume 28, a second vacuum or drawdown pressure may be achieved within inner volume 28 that has a greater magnitude than the first vacuum or drawdown pressure.

As shown in FIGS. 5 and 6, the member 42a and the member 42b are removable from an exterior or from outside the oxygen scavenging dressing 10. A tab or a portion of adhesive member 42a and adhesive member 42b can protrude or extend outwards (e.g., through the film 26) so that a caregiver or a user can grasp the tab. The caregiver or user can pull adhesive member 42a in direction 46 (e.g., along axis 34) and adhesive member 42b in direction 48 (e.g., along axis 34 in an opposite direction), thereby removing adhesive member 42a and adhesive member 42b from the oxygen scavenging dressing 10 so that the first pocket 40a and/or the second pocket 40b are fluidly coupled with the inner volume 28.

It should be understood that while the oxygen scavenging dressing 10 as described herein with reference to FIGS. 5 and 6 only includes two members 42, the oxygen scavenging dressing 10 may include any number of members 42 that are selectably fluidly coupled with inner volume 28. Further, the members 42 are shown as being independently fluidly coupled with inner volume 28 (e.g., in parallel with inner volume 28). In some embodiments, the members 42 are stacked on top of each other to form a series of pockets that are selectively coupled with each other (e.g., in series) and with inner volume 28.

### Dressing Changes

Referring to FIGS. 1-2, oxygen scavenging dressing 10 can include one or more indicators that are viewable by a caregiver from an external position of oxygen scavenging dressing 10. The indicators may be fluidly responsive or fluidly activated. For example, the indicators can be positioned within inner volume 28 but viewable through film 26. The indicators may be positioned between film 26 and an outer or exterior surface of the oxygen scavenging layer 18. Fluid or liquid that is absorbed into oxygen scavenging layer 18 may activate or change (e.g., a color, an opacity, a transparency, etc.) of the indicators. In some embodiments, the indicator changes color to indicate that oxygen scavenging dressing 10 has become full or saturated with liquid. When the oxygen scavenging dressing 10 becomes full or saturated with liquid, the indicator can change color, which is viewable through film 26 to notify the user or caregiver that dressing 10 should be changed.

In some embodiments, film 26 itself is fluidly activated or responsive. Film 26 may have a variable transparency or opacity so that when oxygen scavenging dressing 10 is saturated with liquid or becomes full, an opacity or transparency of film 26 changes. For example, film 26 may be opaque when not in direct contact with liquid or fluid, and may become transparent or at least partially transparent when film 26 directly contacts liquid or fluid. When oxygen scavenging dressing 10 is full with liquid or fluid or becomes completely saturated (as indicated by the fluidly responsive or fluidly activated indicators or film), the caregiver may replace oxygen scavenging dressing 10 with a new oxygen scavenging dressing.

### Process

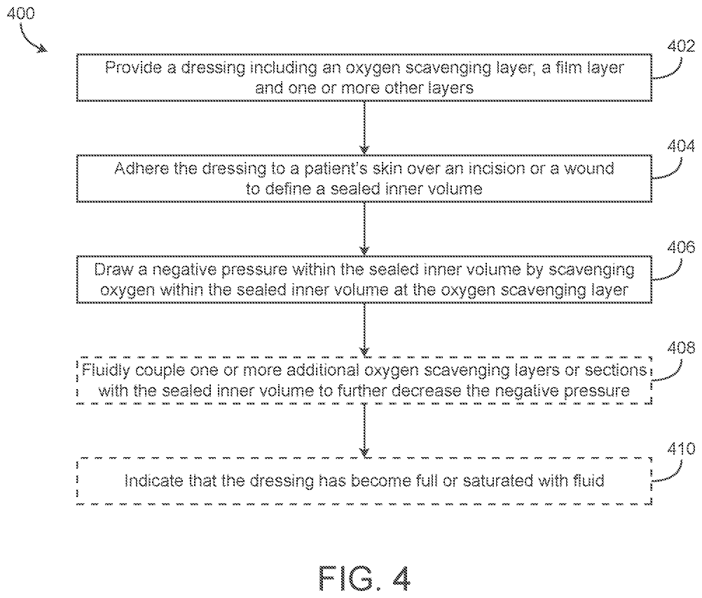

Referring now to FIG. 4, a process 400 for using an oxygen scavenging dressing (e.g., oxygen scavenging dressing 10) is shown, according to some embodiments. Process 400 includes steps 402-410 and can be performed by a caregiver and oxygen scavenging dressing 10. Process 400 facilitates a passive dressing that is low profile and does not require an active pumping or negative pressure system (e.g., a negative pressure system that uses tubular members and a pump to draw a negative pressure at a wound site). The dressing of process 400 can be used for a patient's face, or another area of a patient's body that is highly visible and where an active negative pressure system is not feasible.

Process 400 includes providing a dressing including an oxygen scavenging layer, a film layer, and one or more other layers (step 402), according to some embodiments. Step 402 can include providing oxygen scavenging dressing 10. The oxygen scavenging layer may be oxygen scavenging layer 18 that is configured to react, scavenge, or remove oxygen from within an inner volume of the oxygen scavenging dressing. The film layer can be configured to form an enclosure, an inner volume, a compartment, etc., within which the scavenging layer and the one or more layers are positioned. The film layer may extend over the scavenging layer and the one or more layers and can adhere with or seal with the patient's skin surrounding the wound or the incision. The one or more layers may include patient interface layer 24, absorbent layer 22, and foam layer 20.

Process 400 includes adhering the dressing to the patient's skin over an incision or a wound to define a sealed inner volume (step 404), according to some embodiments. Step 404 can be performed by a caregiver and can be performed by placing the dressing over the incision or the wound so that an underside or an adhesive side of the film layer can adhere with or seal with periwound tissue or periwound skin. In some embodiments, the sealed inner volume is inner volume 28.

Process 400 includes drawing a negative pressure within the sealed inner volume by scavenging oxygen within the sealed inner volume at the oxygen scavenging layer (step 406), according to some embodiments. Step 406 can be performed by the oxygen scavenging layer (e.g., oxygen scavenging layer 18). Step 406 can include scavenging, consuming, reacting with, etc., oxygen in the sealed inner volume. Step 406 may include an oxidation reaction occurring due to the presence of the oxygen scavenging layer.

Process 400 includes fluidly coupling one or more additional oxygen scavenging layers or sections with the sealed inner volume to further decrease the negative pressure (step 408), according to some embodiments. In some embodiments, step 408 is optional. Step 408 can be performed by a caregiver by removing one or more barriers between the one or more additional oxygen scavenging layers or sections and the sealed inner volume.

Process 400 includes indicating that the dressing has become full or saturated with fluid (step 410), according to some embodiments. Step 410 may be performed by a fluidly responsive indicator, layer, or film (e.g., the film layer). Step 410 can include changing a color or an opacity of a fluidly responsive or activated member of the dressing that is viewable by the caregiver so that the caregiver is notified that the dressing has become full or is saturated with fluid. In this way, the caregiver can be notified when the dressing should be changed.

### Configuration of Exemplary Embodiments

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, and/or illustrations of possible embodiments (and such terms are not intended to connote that such embodiments are necessarily extraordinary or superlative examples).

The term “coupled,” as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. Such members may be coupled mechanically, electrically, and/or fluidly.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

The hydrophobicity of a material may vary, but to be considered hydrophobic, generally the material can have an average contact angle with water of at least 90 degrees in some embodiments. To be considered hydrophilic, generally the material can have a contact angle of most 90 degrees in some embodiments. In some embodiments the contact angle with water can be no more than 150 degrees. For example, in some embodiments, the contact angle of the hydrophobic material may be in a range of at least 70 degrees to about 120 degrees with an average contact angle of at least 90 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of a material herein may be further enhanced with a hydrophobic coating of other materials, such as silicones and fluorocarbons, and by any means known, such as by coating by the other material (e.g., coated using a liquid that may be subsequently dried on the material) or plasma coated.

A hydrophobic material can be any material having a solubility in water of less than 10 mg/L at standard temperature and pressure. A hydrophilic material can be any material having a solubility in water of 10 mg/L and greater at standard temperature and pressure.

It is important to note that the construction and arrangement of the wound dressing with optional status indicator as shown in the various exemplary embodiments is illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. Other substitutions, modifications, changes, and omissions may be made in the design, operating conditions and arrangement of the exemplary embodiments without departing from the scope of the present disclosure.

1. An oxygen scavenging dressing, comprising:
a patient interface layer;
an absorbent layer;
a foam layer;
an oxygen scavenging layer; and
a film layer configured to seal with a patient's skin and define an inner volume within which the patient interface layer, the absorbent layer, the foam layer, and the oxygen scavenging layer are positioned.

2. The oxygen scavenging dressing of claim 1, wherein the oxygen scavenging layer is configured to remove oxygen from within the inner volume of the oxygen scavenging dressing to draw a negative pressure within the oxygen scavenging dressing.

3. The oxygen scavenging dressing of claim 1, wherein the oxygen scavenging dressing is configured to be positioned on a patient's face.

4. The oxygen scavenging dressing of claim 1, wherein the film layer comprises an adhesive along an underside configured to seal with the patient's skin.

5. The oxygen scavenging dressing of claim 1, wherein the patient interface layer abuts the patient's skin, wherein the absorbent layer is positioned directly between the foam layer and the patient interface layer, and wherein the foam layer is positioned directly between the oxygen scavenging layer and the absorbent layer.

6. (canceled)

7. (canceled)

8. The oxygen scavenging dressing of claim 1, wherein the oxygen scavenging layer comprises a chemical substance configured to remove oxygen from within the inner volume.

9. The oxygen scavenging dressing of claim 8, wherein the chemical substance is an iron compound.

10. The oxygen scavenging dressing of claim 1, wherein the oxygen scavenging layer comprises two or more sections configured to be selectively deployed to customize a negative pressure capability of the oxygen scavenging dressing.

11. An oxygen scavenging dressing comprising:
an oxygen scavenging layer;
one or more additional layers; and
a film layer configured to seal with a patient's skin and define an inner volume within which the oxygen scavenging layer and the one or more additional layers are positioned;
wherein the oxygen scavenging layer is configured to remove oxygen from within the inner volume of the oxygen scavenging dressing to draw a negative pressure within the oxygen scavenging dressing.

12. The oxygen scavenging dressing of claim 11, wherein the one or more additional layers comprise a patient interface layer, an absorbent layer, and a foam layer.

13. The oxygen scavenging dressing of claim 11, wherein the oxygen scavenging dressing is configured to be positioned on a patient's face.

14. The oxygen scavenging dressing of claim 11, wherein the film layer comprises an adhesive along an underside configured to seal with the patient's skin.

15. The oxygen scavenging dressing of claim 12, wherein the patient interface layer abuts the patient's skin.

16. The oxygen scavenging dressing of claim 12, wherein the absorbent layer is positioned directly between the foam layer and the patient interface layer.

17. The oxygen scavenging dressing of claim 12, wherein the foam layer is positioned directly between the oxygen scavenging layer and the absorbent layer.

18. The oxygen scavenging dressing of claim 11, wherein the oxygen scavenging layer comprises an iron compound configured to remove oxygen from within the inner volume.

19. (canceled)

20. The oxygen scavenging dressing of claim 11, wherein the oxygen scavenging layer comprises two or more sections configured to be selectively deployed to customize a negative pressure capability of the oxygen scavenging dressing.

21. A method for passively drawing a negative pressure at a dressing, the method comprising:
providing a dressing including an oxygen scavenging material, a film layer, and one or more other layers;
adhering the dressing to a patient's skin over an incision or a wound to define a sealed inner volume; and
drawing a negative pressure within the sealed inner volume by scavenging oxygen within the sealed inner volume at the oxygen scavenging material.

22. The method of claim 21, further comprising:
selectably fluidly coupling one or more additional sections of oxygen scavenging material with the sealed inner volume to further decrease the negative pressure.

23. The method of claim 21, further comprising:
selectively deploying two or more sections to customize a negative pressure capability of the dressing.
